(19) United States
(12) Patent Application Publication
(10) Pub. No.: US 2017/0130582 A1
(43) Pub. Date: May 11, 2017

(54) ZERO EMISSION POWER PLANT WITH CO2 WASTE UTILIZATION

(71) Applicant: Ztek Corporation, Woburn, MA (US)

(72) Inventor: Michael S. HSU, Lincoln, MA (US)

(21) Appl. No.: 15/406,142

(22) Filed: Jan. 13, 2017

Related U.S. Application Data
(63) Continuation of application No. 14/115,231, filed on Jan. 24, 2014, now abandoned.
(60) Provisional application No. 61/635,176, filed on Apr. 18, 2012.
(60) Provisional application No. 61/515,900, filed on Aug. 6, 2011.
(60) Provisional application No. 61/482,495, filed on May 4, 2011.

(51) Int. Cl. F01B 21/04; C01B 3/48; C07C 41/09; B01J 19/24; C10G 2/00; C07C 29/152; C01B 3/38; C01B 3/56

(52) U.S. Cl. CPC F01B 21/04; C01B 3/38; C01B 3/386; C01B 3/48; C01B 3/56; B01J 19/245; B01J 19/249; C10G 2/32; C07C 29/152; C07C 41/09; C01B 2203/0261; C01B 2203/0244; C01B 2203/0233; C01B 2203/0283; C01B 2203/042; C01B 2203/0495; C01B 2203/047; C01B 2203/0475; C01B 2203/061; C01B 2203/0833; C01B 2203/1241; C01B 2203/84; B01J 2219/24

(57) ABSTRACT

A clean energy system, a renewable energy system or a zero emission energy system (ZEES) to utilize CO₂ waste. The energy system may include a fuel processor, an energy catalytic reactor, and a power generator. The fuel processor may catalytically convert the CH₄ component in the natural gas, biogas or syngas into a reformate including H₂, CO, CO₂ and H₂O species. The energy reactor may convert the reformate in gas form into a liquid fuel. The power generator may generate power using an output of the fuel processor and/or an output of the energy reactor.

### RELATED APPLICATION

This application is a Continuation application of U.S. application Ser. No. 14/115,231 filed Jan. 24, 2014, which is a 371 national phase application of International Application PCT/US2012/036640 filed May 4, 2012, which claims priority to U.S. Provisional Application Nos. 61/635,176 filed Apr. 18, 2012, 61/482,495 filed May 4, 2011, and 61/515,900 filed Aug. 6, 2011, the contents of which are hereby incorporated by reference.

### BACKGROUND OF THE INVENTION

This application relates to a clean energy system, a renewable energy system or a zero emission energy system (ZEES), and more particularly to a chemical principle based power plant (CPPP) with CO₂ waste utilization.

The governmental efforts to curb greenhouse gas (GHG) emissions have been relying on the laws of individual countries and states for some time now. In general, most countries and states share the interim goal of reducing GHG emissions to 25% below 1990 levels by 2020, and an overall goal of 80% by 2050. However, it is reported that the goals are unlikely to be met by a large margin unless revolutionary technologies are developed in the near future.

When petro-fuel is used with energy systems, carbon credits may be calculated and applied to finance the project due to favorable system hardware characteristics for emission reduction. When renewable fuel is employed, additional carbon credits are calculated due to fuel characteristics. Thus, the potential exits for compounding carbon credits associated with renewable fuel systems, which may be viewed as such systems having a negative CO₂ footprint.

The clean technology may also receive renewable energy credits (REC) administered by agencies of respective countries and states. The REC may only be applied based on the type of fuel used. Fuel cell systems, judged according to their operational principles, do not have renewable characteristics.

### SUMMARY OF THE INVENTION

The present application provides a clean energy system, a renewable energy system or a zero emission energy system (ZEES) with CO₂ waste utilization. This application utilizes chemical principles for concurrent power generation and energy conversion to eliminate CO₂ emissions from power plants and introduce new sources of liquid fuel for transportation. The energy system provided in the present application solves the two most serious problems in the current energy industry: petro-fuel shortage and global warming due to greenhouse gas (GHG) emissions. In one embodiment, the clean energy system is a zero emission energy system when a carbon containing stream with a matched amount of H₂ is used for liquid fuel production and H₂ alone is used for power generation. When employing a renewable feedstock, the energy system constitutes negative CO₂ footprints thus potentially gaining double carbon credits.

According to one practice of the present invention, clean power is generated and any greenhouse gas (GHG) produced by the system is retained and employed to help produce high valued fuels. The energy system provided in the present application may be properly sized for any installation, converting available municipal solid waste (MSW) or biomass into clean energy at affordable costs and with low carbon footprints. The energy system may be applied equally to feedstock including coal and other solid hydrocarbon fuels.

In one embodiment, the energy system may include a fuel processor, an energy catalytic reactor, and a power generator. The fuel processor catalytically converts the CH₄ component in the natural gas, biogas or syngas into a reformate including H₂, CO, CO₂ and H₂O species. The energy reactor may convert the reformate in gas form into a liquid fuel. The power generator may generate power using an output of the fuel processor and/or an output of the energy reactor.

In an embodiment, the fuel processor may include a partial oxidation, autothermal and steam methane reformer. The reformate may be processed with a water shift process to have different percentages of CO vs. CO₂ with a variation range between about 0% and about 20%. The reformate of the fuel processor may be processed with a pressure swing adsorption process to form two flow streams: 1) high purity H₂ steam; 2) high concentration of carbon (CO, CO₂) contents. The CO₂ may be processed with the water shift process to the maximum level of 20%, and processed through the pressure swing adsorption to result in a concentrated CO₂ stream. The concentrated CO₂ stream may be applied for Enhanced Oil Recovery (CO₂-EOR) on-site at oil wells.

In an embodiment, the H₂, CO and CO₂ produced from the fuel processor may be processed in the energy reactor into methanol (CH3OH) in liquid form with a methanol synthesis catalyst. The methanol may be further processed in the energy reactor into DME (CH3OCH3) in liquid form with a suitable catalyst. The DME may be further processed in the catalytic energy reactor into gasoline in liquid form with another suitable catalyst. The mixture of H₂, CO₂ and CO derived from the renewable feedstock may be carried out for producing jetfuel, propane, diesel or heavy liquid fuel production due to high concentration of CO₂, co in the syngas.

According to the teachings of the present invention, the clean energy system may be operated to maximize the use of carbon for the production of a) liquid bio-fuels, including methanol, ethanol, propanol and butanol, as well as b) liquid synfuels, including DME, gasoline, propane, butane, jet fuel and diesel. The energy reactor utilizes chemical-catalysts or bio-catalysts. The energy reactor may be a chemical-catalyst bed, such as a fixed bed, structured bed, slurry bed or microchannel with integrated heat exchanger. The energy reactor may employ single functional catalysts, bifunctional catalysts or multi-functional catalysts to achieve improved performances. The energy reactor may employ bio-catalysts including varieties of yeasts, bacteria and enzymes.

In an embodiment, the reformate may be applied for power generation in one of the following fuel cell electric generators including solid oxide fuel cell (SOFC), molten carbonate fuel cell (MCFC), proton exchange membrane fuel cell (PEMFC), phosphoric acid fuel cell (PAFC) and alkaline fuel cell (AFC). The SOFC is applied in one of three devices: a solid oxide fuel cell; a hybrid system composed of a solid oxide fuel cell and gas turbine unit; a hybrid system composed of a solid oxide fuel cell and a steam turbine unit. The reformate may be applied for power generation in one of the following generators including an internal combustion engine, gas turbine, or steam turbine.

In an embodiment, the reformer may be a Hybrid Reforming System involving electric energy input obtained from a Photovoltaic (PV) panel, wind or tidal wave to meet the energy demands for the reformer's endothermic thermal input and the reformate compression process. The clean energy system may be a Hybrid System involving electric energy input obtained from the PV, wind and tidal wave to support the energy demands for the system, as well as provide remedy for the intermittency of these power sources with storage capability in the form of liquid fuel of the production.

In an embodiment, the clean energy system may be applied to use renewable feedstock that includes municipal solid waste, municipal sewage, farm animal waste, biomass and woody biomass with a feedstock processor. The municipal sewage and farm animal waste may be first processed through digester to yield biogas, which includes methane CH₄, CO₂ and CO. The municipal solid waste, farm biomass, woody biomass may be first processed through gasifier to yield syngas which includes methane CH₄, CO₂ and CO.

In an embodiment, the feedstock processor may be a syngas generator, including a thermal driven, plasma or microwave driven gasifier. The feedstock processor may be a syngas generator, classified as a volatilizer referred by Hathaway Renewable Energy, Inc. of Tennessee. The volatilizer provides syngas of CH₄ rich having heating value exceeding 500 Btu/ft3, other than a gasifier of a common choice providing syngas of H₂ rich with heating value typical of 300 Btu/ft3. The biomass feedstock may include forest waste, community (municipality) waste, coal of solid phase which passing through the volatilizer yields syngas with the heating value over 500 Btu/ft3 composed of CH₄, H₂, CO, CO₂ and other secondary species and biochar or charcoal as the solid residual. The biochar has commercial value as the activated charcoal for commercial use; and claiming for carbon credits and renewable energy credits.

In an embodiment, the biomass feedstock may include waste of liquid phase or sludge which passing through the volatilizer yields syngas with the heating value over 500 Btu/ft3, composed of CH₄, H₂, CO, CO₂ and other secondary species of solid residual. The volatilizer may process the biomass under physical step without undergoing combustion, rather undergoing heating by external sources. The external heating source may be derived from the recycled portion of the syngas, which provides clean burn high temperature source of heat above 1000° C. The external heating source may be derived from the high temperature waste stream, 800° C. to 1000° C., of the SOFC power generator or the high temperature waste stream, 600° C. to 800° C., of the hybrid power generator. The volatilizer operates at a temperature within the above range of the heat sources.

### BRIEF DESCRIPTION OF THE DRAWINGS

The foregoing and other objects, features and advantages of the invention will be apparent from the following description and apparent from the accompanying drawings, in which like reference characters refer to the same parts throughout the different views. The drawings illustrate principles of the invention.

FIG. 1 is a block diagram illustrating clean technology applications including the embodiments provided in the present application.

FIG. 2 is a block diagram of an exemplary clean energy system or zero emission energy system (ZEES) with CO₂ waste utilization in an illustrative embodiment according to the teachings of the present invention.

FIG. 3 is an overview of a zero emission energy system (ZEES) provided according to the teachings of the present invention.

FIG. 4 shows the structure of an exemplary reformer that may be employed in the zero emission energy system (ZEES) depicted in FIG. 3.

FIG. 5 shows the structure of an exemplary energy reactor that may be employed in the zero emission energy system (ZEES) depicted in FIG. 3.

FIG. 6 depicts another embodiment of an exemplary clean energy system according to the teachings of the present invention supporting solar power storage of FIG. 7 and CO2-EOR for oil recovery of FIG. 8.

FIG. 7 is a block diagram of an exemplary energy system supporting Photovoltaic (PV) solar power storage for local grid use in an illustrative embodiment.

FIG. 8 shows an exemplary CO₂-Enhanced Oil Recovery (EOR) system provided in an exemplary embodiment.

### DESCRIPTION OF ILLUSTRATED EMBODIMENTS

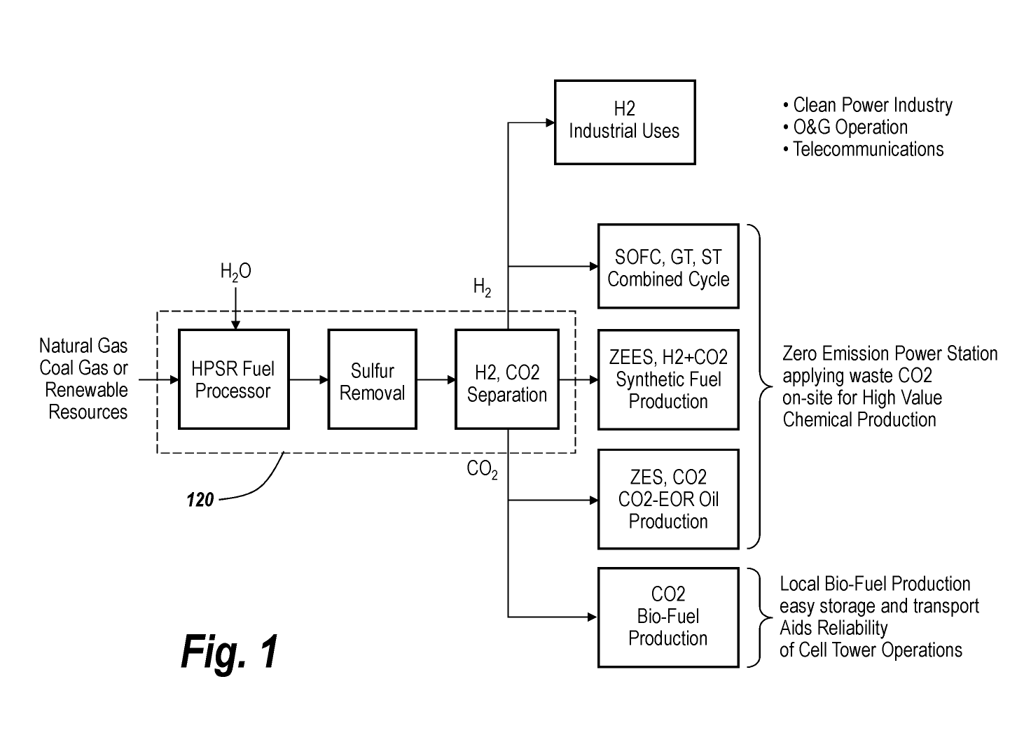

FIG. 1 is a block diagram illustrating clean technology applications including the embodiments provided in the present application. A fuel processor 120 may receive natural gas, coal gas or renewable gas and process the gas to produce, H₂, CO₂ and/or a mixture of H₂ and CO₂. A sulfur component may be removed, and H₂ and CO₂ may be separated.

The produced H₂ may be used for a solid oxide fuel cell (SOFC) system. Fuel cells produce clean exhaust without SOx or NOx through an electrochemical process rather than a thermodynamic process as used in traditional combustion systems. The SOFC also has an advantage in that its clean exhaust remains at a high temperature, which is suitable to drive a traditional system for additional power generation. The overall exhaust remains clean while the power output or the system efficiency doubles.

The SOFC may be integrated with heating, ventilation and air conditioning (HVAC) systems to perform functions for human comfort using the hot exhaust from the SOFC. When energy cost rises, the SOFC-HVAC system may be made available to serve families and communities with a single system performing all necessary power generation functions.

A Zero Emission System (ZES) is employed to make use of CO₂ from the HECP system, in which the hydrogen is fully utilized while the CO₂ is exhausted to the air as unwanted emissions. CO₂ is produced in the HECP system in a concentrated stream for subsequent collection. In one embodiment, the CO₂ gas may be used for injection into an oil field or well to enhance oil production. The CO₂ gas may be compressed for CO₂-EOR (Enhanced Oil Recovery). This may provide opportunities for all small and medium field owners to enjoy the benefits of CO₂ in their wells, in areas of absence of natural CO₂ supply services typically available only to large field owners. The well-known technique of horizontal drilling when coupled with CO₂ injection may provide a deeper and wider reach into the earth with further benefits in thorough sweeping of the sand grains by CO₂ action for enhanced oil production.

An embodiment of the present invention provides a zero emission energy system (ZEES) for eliminating the CO₂ emissions from the traditional power plants and introducing new sources of liquid fuel for transportation. The embodiment utilizes chemical principles for concurrent power generation and energy conversion. The fuelstock is first treated before the power generation to assure clean emissions. The hydrocarbon fuel, either from a petro-source or from a bio-source, is processed following the steam methane reformer reaction into H₂, CO, CO₂ and H₂O, and collectively constitute the reformate. The reformate may be separated into two gas streams: 1) H₂ stream; 2) carbon containing stream. The H₂ stream is primarily utilized for power generation that emits only H₂O (e.g., water molecules). The carbon stream when combined with the proper amount of H₂ from the H₂ stream may be catalytically reacted to form liquid fuels, such as methanol (CH3OH). Other liquid fuel species may be further derived with suitable catalysts. The energy system may provide solutions to simultaneously cure the problems in the current energy industry: petro-fuel shortage and global warming due to greenhouse gas (GHG) emissions. In the energy system, the clean power is generated and the greenhouse gas CO₂ is retained and applied for the production of high valued fuels.

The ZEES provides the energy industry the following benefits: ZEES provides storage solutions to the Solar Electric projects in liquid chemicals; ZEES provides sweeping solution to power generation of emissions issues; ZEES promotes global oil production to relief international political worries; ZEES as GTL facilitates the natural gas move in convenient liquid forms; ZEES as BTL facilitates the renewable bio-feedstock to become commercial energy source; ZEES as a chemical reactor to process petro fuel or biofuel with equal effectiveness; ZEES hybridizing with renewable electricity for storage better serve the future Smart Grid; ZEES thus provides an unhurried transition to better fuel choices.

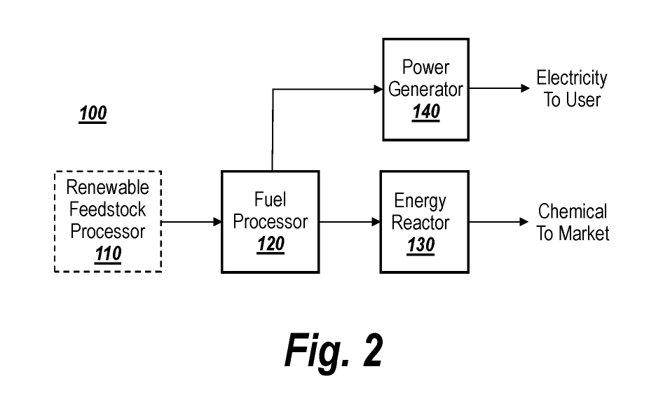

FIG. 2 is a schematic block diagram of an exemplary clean energy system or zero emission energy system (ZEES) with CO₂ waste utilization according to the teachings of the present invention. The ZEES is uniquely qualified for distribution or installation near energy source, market place, and with product shippable for off-site consumption. In the description of the present application, the zero emission energy system refers to an energy system that processes an input feedstock, such as a natural gas or a renewable gas, and produces power with substantially zero emissions of CO and CO₂ gases to the atmosphere. The CO and CO₂ gases produced by processing the input feedstock can be utilized to produce a liquid fuel. The CO and CO₂ gases produced by processing the input feedstock can also be injected into a wellhead to enhance oil recovery.

The energy system 100 may include a fuel processor 120, such as a reformer, an energy catalytic reactor 130, and a power generator 140. The fuel processor 120 can be a reformer that converts a renewable feedstock, such as methane rich gas, into a reformate including H₂, CO and CO₂, which in turn can be introduced to a fuel cell, such as a solid oxide fuel cell (SOFC). Within the reformer, any sulfur present in the reactant may be removed. Reactant pressure may be adjusted. The water may be treated for deionization to prevent corrosion to the equipment in the system and to precondition the reactant for clean emissions. The reformate outputted by the fuel processor 120 may be processed into two separate streams, the H₂ rich stream and the carbon containing CO and CO₂ stream.

The energy catalytic reactor 130 converts H₂ with CO and CO₂ into liquid form of chemical under catalytic reaction according to well-known techniques. Methanol has been recognized as a fundamental liquid chemical derived from syngas or biogas with a commercially available methanol catalyst reactor. Methanol may be further processed into various levels of alcohols, such as ethanol, pentanol, butanol and various commercial fuels such as gasoline, jet fuel and diesel. Gaseous fuel species are difficult to transport or distribute from production plants to the market place. Thus, it is desirable to be able to convert it into liquid form for storage as well as for shipping to market. It also gains the advantage of being used as the fuel for transportation, or vehicle fuels.

The power generator 140 may use the H₂ rich stream derived from the reformer 120 to generate power, a byproduct of which is water. It is a desirable and efficient mode for power generation. The traditional power generators, such as internal combustion engines, gas turbines and steam turbines, may also be powered by the H₂ gas stream. Fuel cells are a suitable generator class on H₂ fuel. The energy system 100 transforms various input gases, such as natural gas, syngas or biogas, into hydrogen rich gas that may be used by the electrochemical reaction of the fuel cell. The Gas Turbine (GT) portion of the hybrid SOFC-GT is capable of reusing the SOFC exhaust to boost the overall efficiency of the combined cycle system to about 70%.

The zero emission energy system (ZEES) may further be applied in renewable applications when a renewable feedstock processor 110 is added prior to the fuel processor 120. Volatilization involves the latest generation of converting organic matter into synthetic gas and biochar. The volatilization process is simpler and cleaner than the previous generation “gasification” technology. Early generation gasification technology uses high temperatures and sometimes high pressure to reformulate matter into the desired components. The process is very complex and has a high parasitic energy cost. Volatilization uses indirect heat in an oxygen starved environment to enable biomass to decompose into methane (45%), CO (15%), H₂ (15%) and other hydrocarbons. The resultant syngas is a very clean product with a LHV greater than 600 Btu/scf which is a factor of two times more energy density than the previous technology. The volatilization units utilized in this embodiment are modular and may be stacked to scale the process in 2.5 MW increments. Each volatilization unit may process 30 tons per day (TPD) of MSW or biomass that contains less than 20% moisture. The volatilizer may also be trailer mounted to produce syngas for transportable requirements. This volatilizer may equally be applied to other feedstock, including coal and all other solid hydrocarbon fuels.

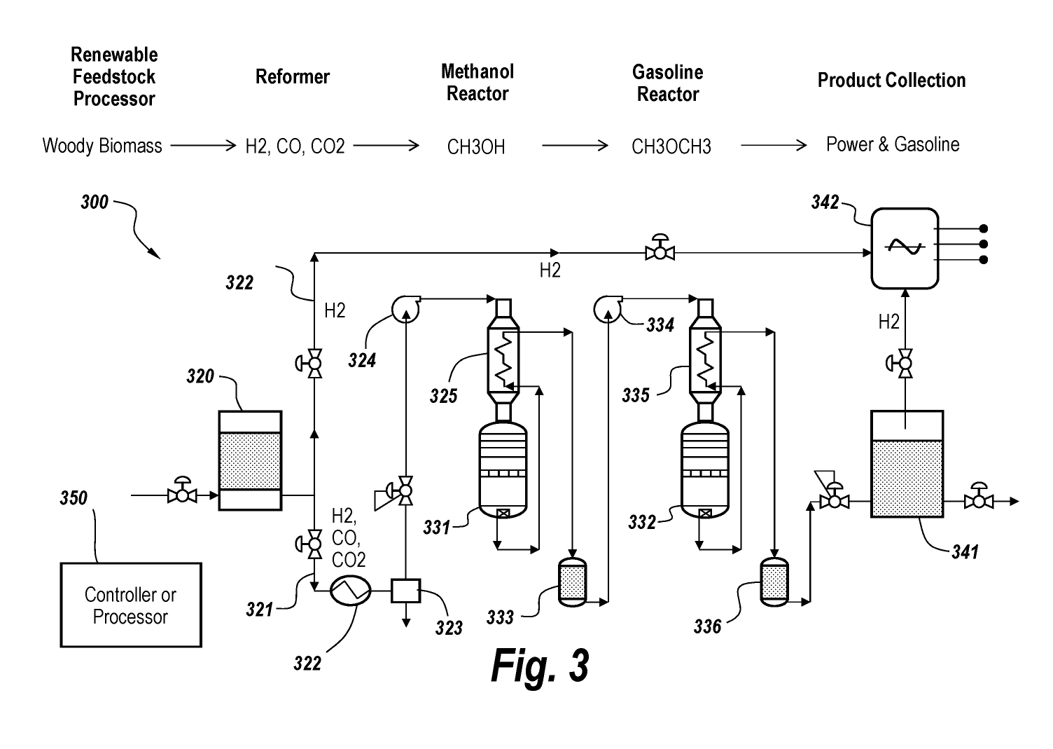

FIG. 3 is a overview of a zero emission energy system 300 provided in an exemplary embodiment. The clean energy system 300 may include a fuel processor 320, energy reactors 331 and 332 and a power generator 342. The fuel processor 320 may be a reformer system that catalytically breakdown the CH₄ component in the fuel source, such as natural gas, biogas or syngas, into H₂, CO, CO₂ and H₂O species joining the output flow and collectively is called the reformate. A power generation branch 322 feeds a fuel reactant for power generation with fuel cells or other traditional generators or engine forming the power generator 342. The energy reactors 331 and 332 are, for example, gas to liquid reactors fed by the liquid chemical production branch 321 of the reformate.

The fuel processor 320 may be a partial oxidation, autothermal or steam methane reformer. The steam methane reformer provides the best performance for pure hydrogen production, if steam supply is unrestricted. A water shift processor following the reformer may be controlled by a processor (not shown) to have a different percentage of CO vs. CO₂ in a range from about 0% to about 20%.

The above reactions are expressed as:

CH₄+H₂O→3H₂+CO Steam Methane Reforming

CO+H₂O→CO₂+H₂ Water Gas Shift Reaction

The reformate may be further processed in a pressure swing adsorption process into two streams of flows: 1) high purity H₂ steam; 2) high concentration of carbon (CO, CO₂) contents.

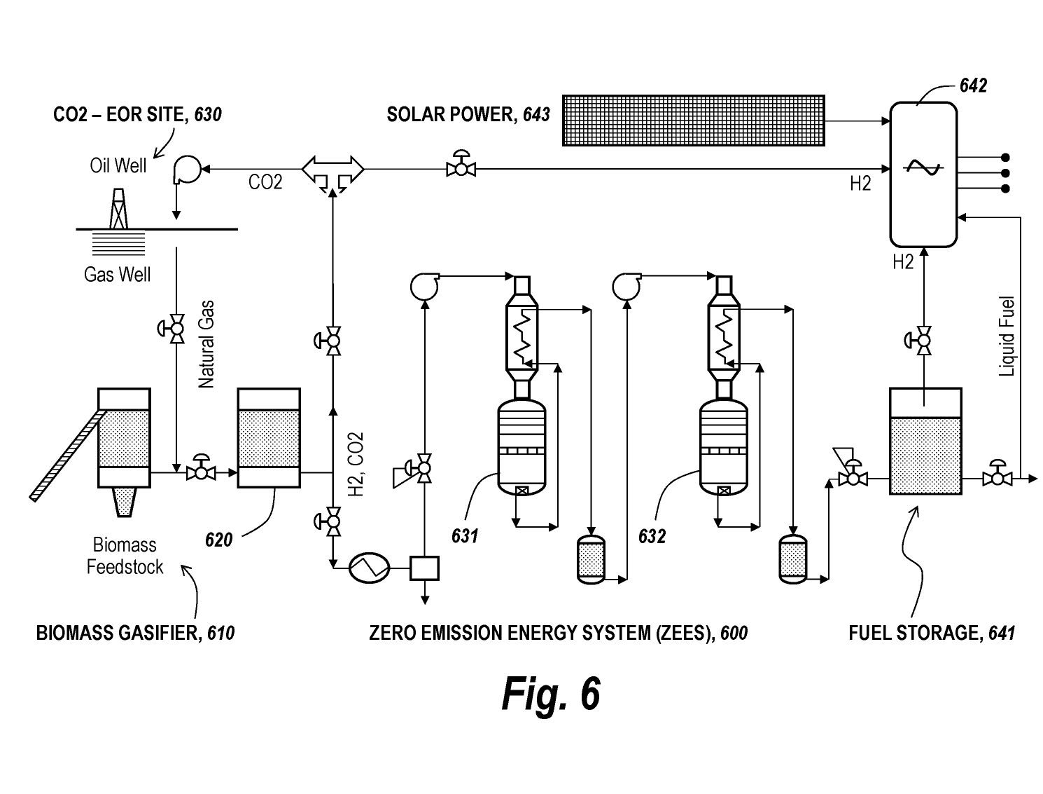

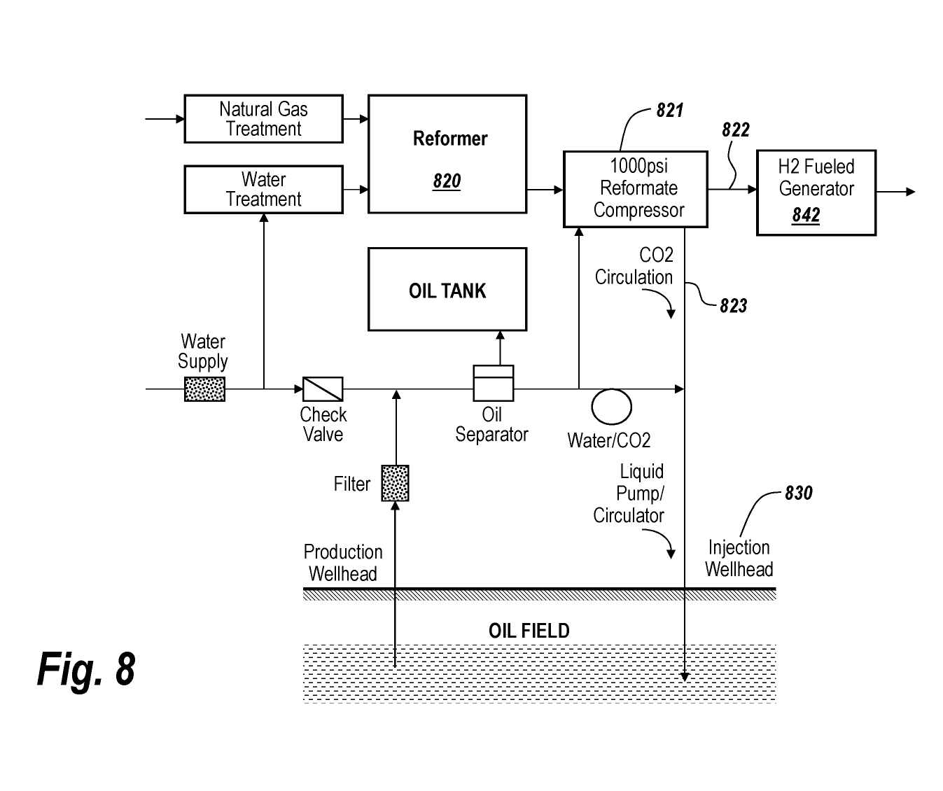

When the reformate is processed in the water shift processor to the maximum level of 20% with low or trace amounts of CO, it may be further processed through the pressure swing adsorption process to yield a concentrated CO₂ stream. The concentrated CO₂ stream may be applied for Enhanced Oil Recovery (CO₂-EOR) on-site at oil wells, as shown in FIG. 6 and FIG. 8.

The liquid chemical production branch 321 carries a mixture of H₂, CO and CO₂ which is introduced into the energy reactor 331 via a heat exchanger 322, a condenser 323, a compressor 324 and a heat exchanger 325 for the production of liquid methanol (CH3OH) with a methanol synthesis catalyst. The condenser 323 may extract water from the reformate and the compressor 324 compresses the reformate before it enters the heat exchanger 325. The methanol may be further introduced into a second energy reactor 332 via a heat exchanger 333, a compressor 334 and a heat exchanger 335 for production of DME (CH3OCH3) in liquid form with a suitable catalyst. The DME as produced may be further processed in a catalytic energy reactor into common gasoline in liquid form with suitable catalyst. The DME or gasoline may be sent to the storage 341 via a heat exchange 336.

The above reactions can be expressed as:

2H₂+CO→CH₄O Methanol Synthesis

2CH₄O→CH₃OCH₃+H₂O Dimethyl Ether (DME) Synthesis

The zero emission energy system 300 may be operated to maximize the use of carbon for the production of, a) liquid bio-fuels, including methanol, ethanol, propanol and butanol, as well as b) liquid synfuels, consisting of DME, gasoline, propane, butane, jet fuel and diesel.

The energy reactor 331 or 332 utilizes chemical-catalysts or bio-catalysts respectively. The energy reactor 331 or 332 employs chemical-catalyst bed in one of the various possible configurations including a fixed bed, structured bed, slurry bed and microchannel with integrated heat exchanger. The microchannel reactor may be of a cylindrical configuration. The energy reactor 331 or 332 may employ single functional catalysts, bifunctional catalysts or multi-functional catalysts to achieve improved performances. The bifunctional or multi-functional catalysts are formed by thorough mixing of basic compounds of individual catalysts, resulting in near instant chained-reactions without unnecessary time lapse. This innovation helps to reduce the size and the number of the reactors, thus leading to cost reduction in system construction. The energy reactor 331 or 332 may also employ bio-catalysts consisting of varieties of yeasts, bacteria and enzymes.

The H₂ stream 322 may be applied for power generation by a power generator 342 with zero CO₂ emission by employing fuel cells or other traditional power generators. The fuel cell electric generators may be selected from one of the types from solid oxide fuel cell (SOFC), molten carbonate fuel cell (MCFC), proton exchange membrane fuel cell (PEMFC), phosphoric acid fuel cell (PAFC) and alkaline fuel cell (AFC). The high performance SOFC has three configurations: a solid oxide furl cell; a hybrid system composed of a solid oxide fuel cell and gas turbine unit; a hybrid system composed of a solid oxide fuel cell and steam turbine unit. The traditional power generation consists of one of the following generators selected from internal combustion engine, gas turbine and steam turbine.

Various pump and valves can be employed throughout the system to help regulate or control the flow of the various fluids. These components can be controlled by a controller or processor 350.

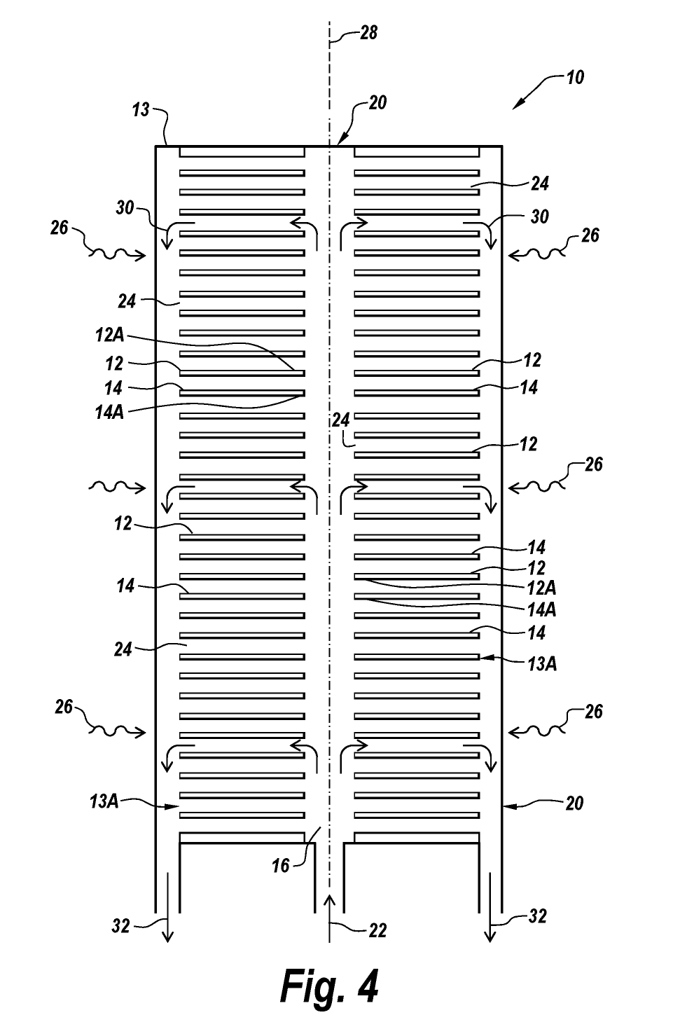

FIG. 4 shows the structure of an exemplary reformer or fuel processor suitable for use in the zero emission energy system (ZEES) depicted in FIG. 3. A reformer 10 may include a stack of thermally conducting plates interspersed with catalyst plates and provided with internal or external manifolds for reactants. The catalyst plate is in intimate thermal contact with the conducting plates so that its temperature closely tracks the temperature of the thermally conducting plate, which may be designed to attain a near isothermal state in-plane to the plate. One or more catalysts may be used, distributed along the flow direction, in-plane to the thermally conducting plate, in a variety of optional embodiments. The reformer may be operated as a steam reformer or as a partial oxidation reformer. When operated as a steam reformer, thermal energy for the (endothermic) steam reforming reaction is provided externally by radiation and/or conduction to the thermally conducting plates. This produces carbon monoxide, hydrogen, steam and carbon dioxide. When operated as a partial oxidation reformer, a fraction of the natural gas is oxidized assisted by the presence of a combustion catalyst and reforming catalyst. This produces carbon monoxide, hydrogen, steam and carbon dioxide. Because of the intimate thermal contact between the catalyst plate and the conducting plates, no excessive temperature may develop within the stack assembly. Details of the plate design may be varied to accommodate a variety of manifolding embodiments providing one or more inlets and exit ports for introducing, pre-heating and exhaust the reactants.

The reformer 10 includes a number of thermally conductive plates 12 and reforming plates 14 that are alternately stacked together to form a stacked reforming structure 13 that extends along axis 28. The reformer includes a fluid conduit 16 that is in fluid communication with the inner portions 12A, 14A of the plates 12, 14. The reformer 10 is preferably housed within a gas-tight enclosure or housing 20. The illustrated reformer may be used to perform both steam and oxidation reforming. The heat necessary for the reforming process may be supplied internally by partial oxidation of hydrocarbon fuel or supplied externally by a remote heat source, as shown by wavy lines 26, to the reformer 10 by radiation, conduction or convection.

The reactant to be reformed by the reformer 10 is introduced into the apparatus through the axial fluid manifold 16. The reactant preferably comprises a mixture of a hydrocarbon fuel and a reforming agent, such as air, oxygen, water or CO₂, that are premixed either prior to introduction to the manifold 16 or within the reformer. The illustrated reformer 10 includes at least one manifold that delivers a fuel/reforming agent mixture to the reformer, rather than provide separate input manifolds for each gas constituent. The introduction of a premixed reactants to the reformer 10 provides for a relatively simple design.

The reactant mixture 22 is introduced to the manifold 16 by any appropriate means, such as by fluid conduits. The mixture 22 enters the inner portions of the reformer through reactant passages 24 that are formed between the adjacent conductive plates 12 and reforming plates 14. The passages may comprise any surface indentation or protrusions, which may be formed by embossing, and which constitutes a substantially continuous fluid passage that extends from the manifold 16 to the outer peripheral surface 13A of the stacked reforming structure 13. The passages may also be formed by utilizing conductive or reforming plates that are made of a porous material or have a power reformer catalyst material coated or formed thereon, thus allowing the reactant to pass through the reformer.

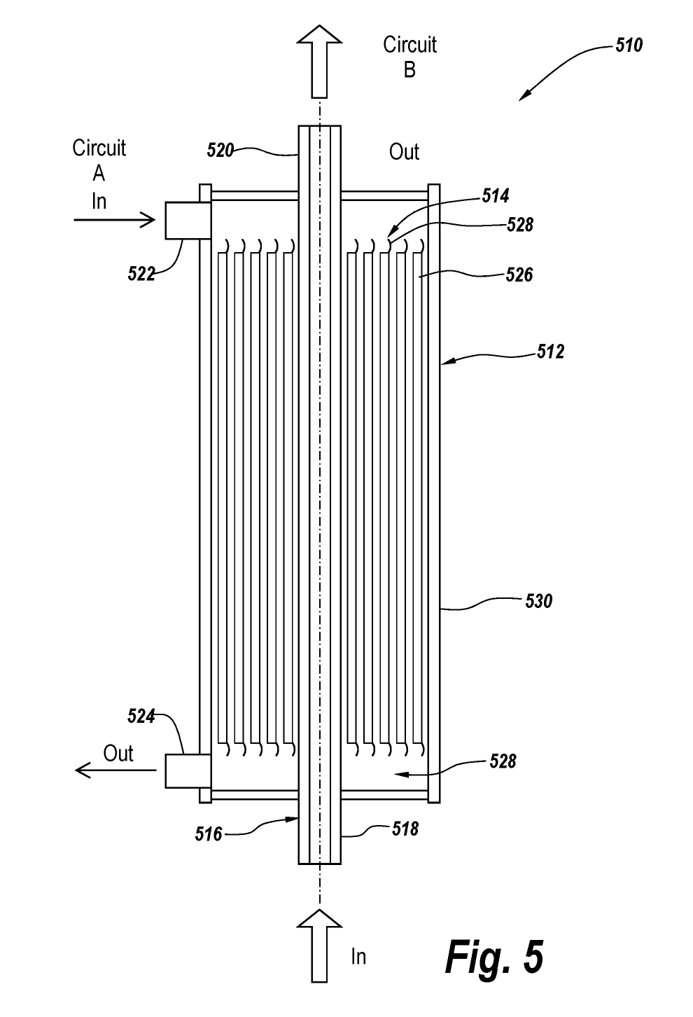

FIG. 5 shows the structure of an exemplary reactor in the zero emission energy system (ZEES) depicted in FIG. 3. The reactor 510 may be a Cylindrical Catalytic Reactor or a Cylindrical MicroChannel (CMC) Reactor that has cylindrical microchannel (CMC) structure to incorporate catalyst to be used as a catalytic reactor. The reactor 510 may include a housing 512 defining a chamber 528 that has an inlet 522 and an outlet 524. The housing can have any selected shape or size, and preferably has a cylindrical. A bundle element 514 is mounted within the chamber 528. The bundle element can include a conduit and a multi-sheet layer 526 which can be wrapped around the conduit. The multi-sheet layer 526 can comprise at least two sheets which define a confined flow volume. The bundle element 514 can also be constructed as a series of tubes. The bundle element 514 can include a conduit 516 that passes through the housing 512 and extends between an inlet 518 and an outlet 520. The inlet and outlet do not communicate directly with the chamber 528.

In an embodiment, the bundle element includes double layers. The space formed between the double layers is designated as the B-side (Circuit B). The space formed between the rolled double layers is designated as the A-side (circuit A). The A-side allows straight flow path along the axis of the reactor with an inflow port 522 and outflow port 524. The B-side presents a spiral flow path between the double layers with an inflow port 518 and outflow port 520. A round screen or perforated plate is placed and secured at the one end (bottom) of the cylinder, which allows the loading of catalyst of proper size to fill the A-side of the reactor when positioned upright for operation. A removable round screen or perforated plate may be placed at the other end (top). The B-side is used as the path for the thermal fluid to flow through for temperature regulation, heating or cooling.

The space between the “double layers” and the space between the “spirally rolled double layers” may be supported by dimples formed in the sheets before welding and rolling. The space may be controlled in millimeter sizes to provide excellent heat transfer capability between the medium on the A-side through the metal layer to the B-side. The rolled configuration presents large surface area to be packaged in small cylindrical volume.

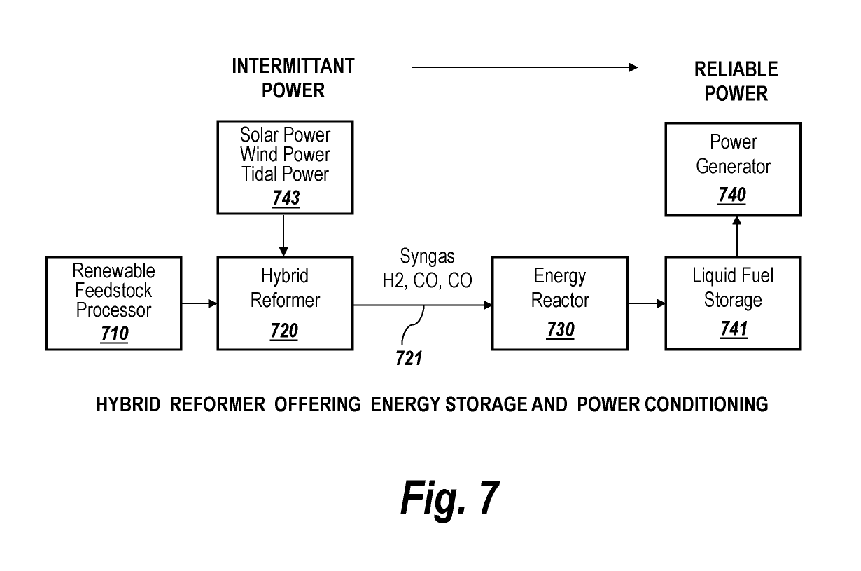

FIG. 6 depicts another embodiment of an exemplary clean energy system 600 according to the teachings of the present invention supporting solar power storage of FIG. 7 and CO2-EOR for oil recovery of FIG. 8. The zero emission energy system 600 may include a fuel process 620, energy reactors 631 and 632, a storage 641 and a power generator 642, which are substantially the same elements as described above with respect to FIG. 3.

The zero emission energy system 600 may be applied to use renewable feedstock that includes municipal solid waste (MSW), municipal sewage, farm animal waste, biomass and woody biomass with a feedstock processor. The municipal sewage and farm animal waste may be first processed through a digester to yield biogas, which includes methane CH4, CO₂ and CO. The municipal solid waste, farm biomass, woody biomass may be processed through a gasifier 610 to yield syngas which includes methane CH₄, CO₂ and CO.

The feedstock processor may be a syngas generator, selected from a thermal driven, plasma or microwave driven gasifier. A desirable syngas generator classified as a volatilizer 610 provides syngas of CH₄ rich having heating value exceeding 500 Btu/ft3, other than a gasifier of a common choice providing syngas of H₂ rich with heating value typical of 300 Btu/ft3. The renewable feedstock includes forest waste, community (municipality) waste, coal of solid phase and even waste of liquid phase or sludge which passing through the volatilizer yields syngas and other secondary species including variable amount of biochar or charcoal as the solid residual.

The volatilizer 610 may process the biomass under physical step without undergoing combustion, rather undergoing heating by external sources. The external heating source may be derived from the recycled portion of the syngas, which provides clean burn high temperature source of heat above 1000° C. The external heating source may also derived from the high temperature waste stream, 800° C. to 1000° C., of the SOFC power generator or the high temperature waste stream, 600° C. to 800° C., of the hybrid power generator.

The biochar left behind in volitilizer 610 has commercial value as the activated charcoal for commercial use and may claim for carbon credits and renewable energy credits.

The clean energy system 600 is a zero emission energy system when carbon containing stream with matched amount of H₂ is used for liquid fuel production and H₂ alone is used for power generation. The system when applied to use renewable feedstock constitutes negative CO₂ footprints thus gaining double carbon credits.

FIG. 7 is a block diagram of an exemplary energy system supporting Photovoltaic (PV) solar power storage for local grid use in an illustrative embodiment. The energy system is provided for the commercial Smart Grid Build-up and as the remedy for PV power intermittency. For low cost production of hydrogen, a Hybrid Reforming System 720 involving electric input obtained from the PV solar power generator 743 is utilized, in conjunction with the use of a renewable biomass to be processed through a renewable feedstock processor 710. The electrical storage mechanism occurs through the energy consumption of the endothermic chemical reforming reaction, steam generation and the reformate compression that constitutes as much as 50% of the energy input for the process. The storage medium may be biomethanol (methanol) or equivalent liquid fuel to be produced in the energy reactor 730 and stored in the liquid fuel storage 741. The power generator 740 can be operated on demand rather than on the unsteady solar supply. The concept as represented in FIG. 7 can be implemented in the ZEES System 600 in FIG. 6.

FIG. 8 shows an CO₂-Enhanced Oil Recovery (EOR) system provided in an exemplary embodiment. As the energy price fast rising, the interest in oil and gas exploration and production is mounting, relying upon advanced stimulation techniques for improved recovery. As discussed above, a reformer system 820 produces a reformate from an input resource, such as a natural gas. A compressor 821 compresses the reformate. The hydrogen 822 may be used in the power generator 842 to generate power. The CO₂ gas 823 may be injected into wellhead 830 on-site field to stimulate the production of oil.

Since the reformer is installed on site, this system can greatly enhance the operational income. Furthermore, the CO₂ sequestration credit may significantly offset the capital cost of the fuel cell or reformer system, which enhances the financial incentive of the adopting this advanced ZEES system 600 in FIG. 6 for stimulating oil and gas production.

As described above, the embodiment in this application utilizes chemical principles for concurrent power generation and energy conversion. The embodiment eliminates the CO₂ emissions from the power plants and introduces new sources of liquid fuel for transportation. Therefore, this application provides solutions to simultaneously cure the two most serious problems in the current energy industry: petro-fuel shortage and global warming due to greenhouse gas (GHG) emissions. In the embodiments, the greenhouse gas is retained and applied for the production of high valued fuels. This invention may be applied equally to renewable feedstock and other feedstock including coal and solid hydrocarbon fuels.

It will thus be seen that the invention efficiently attains the objects set forth above, among those made apparent from the preceding description. Since certain changes may be made in the above constructions without departing from the scope of the invention, it is intended that all matter contained in the above description or shown in the accompanying drawings be interpreted as illustrative and not in a limiting sense.

It is also to be understood that the following claims are to cover generic and specific features of the invention described herein, and all statements of the scope of the invention which, as a matter of language, might be said to fall therebetween.

Having described the invention, what is claimed as new and desired to be secured by Letters Patent is:

What is claimed is:

1. A clean energy system, comprising:
a fuel processor receiving a natural gas, biogas or syngas and catalytically converting a CH.sub.4 component in the natural gas, biogas or syngas into a reformate including only H.sub.2, CO, CO.sub.2 and H.sub.20 species;
a heat exchanger for controlling a temperature of the reformate;
an energy reactor converting the reformate in gas form into a liquid fuel; and
a power generator generating power using an H.sub.2 component from an output of the fuel processor or from an output of the energy reactor,
wherein the system is a zero emission power plant qualified for installation near energy source, market place, and with product shippable for off-site consumption.

2. The energy system of claim 1, wherein the fuel processor comprises a partial oxidation reformer, an autothermal reformer or a steam methane reformer.

3. The energy system of claim 2, wherein the reformate is processed with a water shift process to have different percentages of CO vs. CO.sub.2.

4. The energy system of claim 2, wherein the reformate is processed according to a pressure swing adsorption process to form a H.sub.2 steam and a carbon stream, which includes at least the CO and CO.sub.2.

5. The energy system of claim 1, wherein the CO.sub.2 is processed according to a water shift process, and is processed through a pressure swing adsorption process to form a concentrated CO.sub.2 stream.

6. The energy system of claim 1, further comprising
a condenser disposed between the fuel processor and the energy reactor for extracting water from the reformate, and
a first heat exchanger disposed between the condenser and the fuel processor for exchanging heat with the reformate.

7. The energy system of claim 6, further comprising
a first compressor disposed between the condenser and the energy reactor to compress the reformate output of the condenser,
a second energy reactor disposed between the energy reactor and the power generator for further processing the reformate into a liquid fuel,
a second heat exchanger disposed between the energy reactor and the second energy reactor,
a second compressor disposed between the second heat exchanger and the second energy reactor for compressing the reformate output from the second heat exchanger,
a storage tank for storing the liquid fuel, and
a third heat exchanger disposed between the second energy reactor and the storage tank.

8. The energy system of claim 1, wherein the fuel processor comprises
a plurality of thermally conducting plates, and
a plurality of catalyst plates,
wherein the thermally conducting plates are alternately stacked together with the catalyst plates to for a stack.

9. The energy system of claim 1, wherein the energy reactor comprises
a housing defining a chamber that has an inlet and an outlet, and
a bundle element mounted within the chamber having a conduit that extends between the inlet and the outlet and a flow confining structure disposed about the conduit.

10. The energy system of claim 1, further comprising a renewable feedstock processor coupled to an input of the fuel processor.

11. The energy system of claim 1, wherein H.sub.2, CO and CO.sub.2 is further processed in the energy reactor into methanol (CH3OH) in liquid form with methanol synthesis catalyst, and the methanol is further proceed in the energy reactor into DME (CH3OCH3) in liquid form with suitable catalyst.

12. The energy system of claim 1, wherein a mixture of H.sub.2, CO.sub.2 and CO derived from renewable feedstock is carried out for the production of:
liquid fuels comprising methanol, ethanol, propanol, and butanol; and
liquid synfuels comprising CH.sub.3OCH.sub.3 (DME), gasoline, butane, jetfuel, propane, diesel; or
heavy liquid fuel production due to high concentration of CO, CO.sub.2 in the syngas.

13. The energy system of claim 1, wherein the reactor employs single functional catalysts, bifunctional catalysts or multi-functional catalysts to achieve improved performances.

14. The energy system of claim 1, wherein the system is a Hybrid System involving electric energy input obtained from a photo voltaic panel, wind or a tidal wave to support the energy demands for the system.

15. The energy system of claim 1, wherein the system is a zero emission energy system when carbon containing stream with matched amount of H.sub.2 is used for liquid fuel production and H.sub.2 alone is used for power generation.

16. The energy system of claim 15, wherein when applied to use renewable feedstock constitutes negative CO.sub.2 footprints thus gaining double carbon credits.

17. A method for generating power, comprising:
receiving natural gas, biogas or syngas and catalytically converting a CH.sub.4 component in the natural gas, biogas or syngas into a reformate including only H.sub.2, CO, CO.sub.2 and H.sub.20 species;
controlling a temperature of the reformate using a heat exchanger;
converting the reformate in gas form into a liquid fuel;
storing the liquid fuel in a storage; and
generating power using a hydrogen gas separated from the reformate or a hydrogen gas discharged from a liquid fuel processor.

18. The method of claim 17, wherein the CO.sub.2 separated from the reformate is injected into a wellhead to enhance oil recovery (EOR), simultaneously to achieve a state of zero emission power generation.

19. A method for generating clean power in a system, comprising:
receiving a natural gas, biogas or syngas and catalytically converting a CH.sub.4 component in the natural gas, biogas or syngas into a reformate including only H.sub.2, CO, CO.sub.2 and H.sub.20 species;
controlling a temperature of the reformate using a heat exchanger;
processing the reformate to obtain concentrated CO.sub.2; and
employing at least one of a fuel cell generating power using a Hydrogen output of a fuel processor or a Hydrogen output of a energy reactor, a common internal combustion (IC) engine or gas turbine using the Hydrogen output of the fuel processor or the Hydrogen output of the energy reactor, or a solid oxide fuel cell (SOFC) Hybrid System with a bottoming gas or a steam turbine using the Hydrogen output of the fuel processor or the Hydrogen output of the energy reactor,
wherein the system is a zero emission power plant qualified for installation near energy source, market place, and with product shippable for off-site consumption.

20. The method of claim 19, wherein the concentrated CO.sub.2 extracted from the reformate is injected into a wellhead to enhance oil recovery (EOR), simultaneously to achieve a state of zero emission power generation.
